(19) United States
(12) Patent Application Publication
(10) Pub. No.: US 2005/0058732 A1
(43) Pub. Date: Mar. 17, 2005

(54) Herbal pharmaceutical composition for treating siriasis and its process

(76) Inventor: Wei Xiao, Lianyungang (CN)

Correspondence Address: FISH & NEAVE IP GROUP, ROPES & GRAY LLP, 1251 AVENUE OF THE AMERICAS FL C3, NEW YORK, NY, 10020-1105, US

(21) Appl. No.: 10/903,666

(22) Filed: Jul. 30, 2004

Related U.S. Application Data
(63) Continuation of application No. PCT/CN02/00050, filed on Feb. 1, 2002.

(51) Int. Cl. A61K 35/78; A61K 31/12

(52) U.S. Cl. 424/739; 424/742; 424/756; 424/745; 424/760; 424/779; 514/691

(57) ABSTRACT

The invention relates to a pharmaceutical composition for treating siriasis and its process. The composition is formulated by Camphor, dried ginger, rhubarb, Common fennel, Chinese cinnamon, hot pepper and oleum eucalypti. According to the invention, the pharmaceutical composition can be taken conveniently and absorbed quickly. It has high contents of active ingredient, and accordingly it has a good therapeutic effect.

### FIELD OF THE INVENTION

The invention is related to a kind of composition of traditional Chinese medicine, especially a composition for treating heatstroke and the preparation of the composition thereof.

### BACKGROUD OF THE INVENTION

Heatstroke is a dysfunction of the thermotaxic center and the sudoriferous gland as well as the over loss of the water and the electrolyte caused by hot environment. The clinical manifestations of the heatstroke are dizzy, sick, bellyache and the discomfort of the stomach and intestine. It was recorded in the earliest traditional Chinese medicine works—The Yellow Emperor's Canon of Internal Medicine that the summer-heat evil had many features. It is a kind of yang evil with the feature of ascending and dispersing, and it can consume the qi and the body fluid seriously. The summer-heat evil is always companied by the damp evil so that it can easily obstruct and disorder the movement of the qi and consume the normal yang qi. If the body is attacked by the summer-heat evil, the channels and collaterals turn unfluent and the function of the spleen and stomach become disordered so that the people shows dizziness, sickness, bellyache, stuffy in the chest and discomfort in the belly. According to the mechanism foregoing, the heatstroke should be treated on the method of clearing away heat and eliminating dampness, opening the orifice and harmonizing the stomach, normalizing the movement of the qi. The relevant medicines for treating the heatstroke are still lacking by far and because the tincture normally used is with the high concentration of alcohol that the medicine's taste is bad for drink. What's more, if the patient is allergic to the alcohol, the tincture can't be used. For the reasons above, the current medicine can't be widely used in the clinical.

### THE CONTENT OF THE INVENTION

The aim of the invention is to provide a kind of herb composition. with the features of quicker acting, better curative effect, higher content of the effective ingredient and easier to administrate and the preparation method of this herb composition.

Another aim of the invention is to provide the new use of this composition in the manufacture of medicament. The aim of the invention comes into practice by the technical embodiment as following:

The invented medicine is made from the materials as follows:

| | |
|---|---|
| Camphor | 50-250 part by weight |
| dried ginger | 50-250 part by weight |
| rhubarb | 30-200 part by weight |
| common fennel | 15-100 part by weight |
| chinese cinnamon | 15-100 part by weight |
| hot pepper | 10-60 part by weight |
| oleum eucalypti | 20-120 volume/weight |

The preferred ratio of these materials is that:

| | |
|---|---|
| Camphor | 70 part by weight |
| Dried ginger | 80 part by weight |
| Rhubarb | 40 part by weight |
| Common fennel | 40 part by weight |
| Chinese cinnamon | 50 part by weight |
| Hot pepper | 15 part by weight |
| oleum eucalypti | 100 volume/weight |

The preferred ratio of these materials is that:

| | |
|---|---|
| Camphor | 125 part by weight |
| Dried ginger | 125 part by weight |
| Rhubarb | 100 part by weight |
| Common fennel | 50 part by weight |
| Chinese cinnamon | 50 part by weight |
| Hot pepper | 25 part by weight |
| oleum eucalypti | 62.5 volume/weight |

The preparation technology of the invented medicine is that:

Dried ginger, Chinese cinnamon, common fennel, rhubarb and hot pepper are crushed into crude powder; dried ginger, Chinese cinnamon and common fennel are distilled respectively in the distiller to collect the naphtha for the further use.

The 3 kind of herbs distilled are mixed with rhubarb and hot pepper uniformly and then the mixture are humidified by the 80% alcohol. The humid herbs are percolated by the 70% alcohol 6-10 times weight of the herbs. The alcohol after the percolation are collected and mixed. The alcohol is recycled from the mixed liquor until no alcohol smell can be felt. The new liquor is condensed in the low pressure to thick paste on the relative density of 1.20 at 60° C. The thick paste is dehydrated in the vacuum and then the dried medicine is crushed into fine powder better than the level of 100-mesh with the water content less than 5%.

The camphor, oleum eucalypti and naphtha are added into the refined vegetable oil and the mixed oil is heated in the water bath at 60-70° C. until the camphor is dissolved. When the oil solution turns clear, the fine powder made from the :thick paste is added into the oil. The mixed oil is agitated adequately to prevent the conglomeration of the powder. The mixed oil is adjusted to the right weight by adding refined vegetable oil. The suspended oil, which is made uniformly, is milled by the colloid mill for 2 times to get a stabilized suspension. At last, the suspension is formulated into soft capsules by the machine.

To guarantee the quality and effect of the invented medicine and to avoid the loss the effective ingredients, dried ginger, common fennel and Chinese cinnamon which contain the ingredient of naphtha are distilled respectively by the water to get the naphtha first; oleum eucalypti and camphor are added directly; the extracted paste is dehydrated in the low pressure to avoid the damage of the effective ingredients.

The content of camphor in each capsule is more than 53 mg. The dosage of the invented medicine is 1-2 capsules each time and 3 times a day by oral administration. The capsule of the invention disintegrated within 4-5 minutes. Comparing with the current medicine, the capsule of the invention has the features of quicker acting, better curative effect, lower dose for oral administration and easier to adrministrate.

The capsule of the invention for treating the dizziness, sickness, bellyache and discomfort of the stomach and intestine related to the heatstroke has a definite effect and has been generally accepted by the patients.

### EXPERIMENTAL EXAMPLE I

(I)The experiment animal:

The Kunming white mice were bought from animal center of Beijing Medicine College. The sex was not limited.

(II)The experiment medicine:

The content of the capsule of the invention, each ml containing the crude drug 0.6 g, were diluted by peanut oil or distilled water before the experiment. The castor oil was supplied by the Medicine and Biological products Detection Centre belongs to the National Ministry of Health of China.

(III)The method and result:
1. The effect on the pulsive movement of the small intestine in the mouse

The mice weighting 18-22 g, half male and half female, were divided into 5 groups. Each group had 12 mice. Group 1 were fed with 0.9% NS; group 2 were. fed with peanut oil; group 3 were fed with soft capsule on the concentration of 100 mg/kg; group 4 were fed with soft capsule on the concentration of 400 mg/kg; group 5 were fed with 7% alcohol solution. All the animals were fasted for 10 hours at this period water were supplied. After the 10-hour fast the animals in the different groups were fed with the different medicines on the dosage of 0.1 ml/10 g by the stomach injection. 1 hour after the first stomach injection, all the animals were fed with the 5% active carbon suspension (the suspension were made by 10% gum arabic) by stomach injection. 25 minutes later, all the animals were killed by dislocating the cervical vertebra and their small intestine were taken out to be measured. The dimension 1 (from the pylorus to the appendices) and the dimension 2 (from the pylorus to the place where the carbon reached) were measured and recorded.

$$The carbon propelling ratio \% = \frac{Dimension 2}{Dimension1} \times 100 \%$$

The result indicated that the soft capsule on both concentration of 100 mg/kg and 400 mg/kg and the alcohol could inhibit the movement of small intestine while the peanut oil could not. (see table 1)

TABLE 1
The capsule of the invention's effect on the small intestine
propelling rate in the mouse

| | | propelling rate |
|---|---|---|
| group | number | (\overline{X} ± SD %) |
| 0.9% NS | 12 | 82.7 ± 6.3 |
| peanut oil | 12 | 79.1 ± 8.0 |
| 7% alcohol | 12 | 74.1 ± 10.5* |
| invented capsule 100 mg/kg | 12 | 64.8 ± 9.53**Δ |
| invented capsule 400 mg/kg | 12 | 51.1 ± 12.80**ΔΔ |

note:
comparing with the peanut oil group: ΔP < 0.05 ΔΔP < 0.01
comparing with the alcohol group: ⁰P < 0.05 ⁰⁰P < 0.01

2.The effect on the diarrhea caused by the castor oil in the mouse

The mice weighting 20-22 g, half male and half female, were divided into 3 groups. Each group had 12 mice. Group 1 were fed with 0.9% NS (normal saline); group 2 were fed with soft capsule on the concentration of 100 mg/kg; group 3 were fed with soft capsule on the concentration of 400 mg/kg. All the animals were fasted for 10 hours at this period water were supplied. After the 10-hour fast the animals in the different groups were fed with the different medicines on the dosage of 0.1 ml/10 g by the stomach injection. 1 hour after the first stomach injection, each mouse was fed with 0.2 ml castor oil by stomach injection. The defecation of the mice were observed and recorded every 1 hour in the next 6 hours. The results showed that the capsule of the invention on the concentration of 100 mg/kg and 400 mg/kg could inhibit the diarrhea caused by the castor oil.

TABLE 2
The effect of the capsule of the invention on the diarrhea
caused by the castor oil in the mouse

| | | the diarrhea times |
|---|---|---|
| | | in the 6 hours |
| group | number | (\overline{X} ± SD) |
| 0.9% NS | 12 | 10.0 ± 2.73 |
| invented capsule 100 mg/kg | 12 | 5.08 ± 4.46*ΔΔ |
| invented capsule 400 mg/kg | 12 | 4.25 ± 3.19*Δ |

note:
comparing with the 0.9% NS group: *P < 0.01

1.The effect on the mouse's spontaneous activity

The mice weighting 19-21 g, half male and half female, were divided into 5 groups. Each group had 10 mice. Group 1 were fed with 0.9% NS; group 2 were fed with peanut oil; group 3 were fed with 3.5% alcohol ; group 4 were fed with soft capsule on the concentration of 25 mg/kg; group 5 were fed with soft capsule on the concentration of 100 mg/kg. All the animals in the different groups were fed with the different medicines on the dosage of 0.2 ml/10 g by the stomach injection. In the next 45 minutes after the stomach injection the mice were put into the MK-ANIMEX activity recorder (35×21×15 cm) 2 mice one time to record the activity quantity. The first 3 minutes after put into the recorder were left for the mice to adapt the environments, and in the next 6 minutes the mice's activity quantity were recorded by the machine. The activity quantities were printed by the DP-41 digital printer connected to the MK-ANIMEX activity recorder. The results showed that the capsule of the invention on the concentration 25 mg/kg could improve the mice's activity quantity remarkably, while the other medicine had no remarkable effect on the activity quantity. (see table 3)

TABLE 3
The effect of the invented medicine on the mouse's
spontaneous activity

| | | activity quantity |
|---|---|---|
| | | in 6hours |
| group | number | (\overline{X} ± SD) |
| 0.9% NS | 10 | 697.1 ± 53.82 |
| peanut oil | 10 | 689.7 ± 78.12 |
| 3.5% alcohol | 10 | 727.8 ± 25.46 |
| invented medicine 25 mg/kg | 10 | 785.2 ± 41.34* |
| invented medicine 100 mg/kg | 10 | 709.8 ± 28.93 |

note:
comparing with the 0.9% NS group: *P < 0.01

4. The effect on the sleep time induced by the pentobarbitale sodium

The mice weighting 18-22 g, half male and half female, were divided into 4 groups. Group 1 were fed with 0.9% NS; group 2 were fed with soft capsule on the concentration of 25 mg/kg; group 3 were fed with soft capsule on the concentration of 50 mg/kg; group 5 were fed with soft capsule on the concentration of 75 mg/kg. All the mice were fed with the corresponding medicine on the dose of 0.2 ml/10 g by stomach injection. 45 minutes after the injection, all the mice were injected pentobarbitale sodium into the abdominal cavity on the dosage of 54 mg/kg. The disappearing of righting reflex was adopted as the index of sleep and all the mice's sleep time were recorded. The result showed that the invented medicine could reduce the sleep time on all the three dosage. (see table 4)

TABLE 4
The effect of the capsule of the invention on the sleep
time induced by the pentobarbitale sodium

| | | | sleep time |
|---|---|---|---|
| group | dosage(mg/kg) | number | (min \overline{X} ± SD) |
| 0.9% NS | | 10 | 98.9 ± 23.5 |
| invented capsule | 25 | 12 | 80.1 ± 15.5* |
| invented capsule | 50 | 12 | 75.3 ± 19.5* |
| invented capsule | 75 | 12 | 77.8 ± 17.2* |

note:
comparing with the 0.9% NS group: *P < 0.05

This experiment showed that the capsule of the invention could inhibit the propelling movement of the mice's small intestine, improve the diarrhea caused by castor oil and reduce the sleep time induced by pentobarbitale sodium. The 25 mg/kg soft capsule could improve the spontaneous activity quantity remarkable. The result proved that the capsule of the invention had a definite effect to improve the dizziness, sickness, diarrhea and uncomfort of stomach and intestine caused by heatstroke.

### EXPERIMENTAL EXAMPLE II

(I)Diagnostic criteria:
1. Diagnostic criteria of Traditional Chinese Medicine:

(1)The features in the occurrence of disease: The heatstroke always happens in the summer day with high temperature. The people who work in the high temperature or under the burning sun are easily attacked by summer-heat evil. Sometimes the people who eat too much cold things or stay in an unfit cold place in a summer day can be attacked by summer-heat evil too. If the people have been caught by a cold rain or gone through cold water, they are easily be attacked by the combined evil of summer-heat and damp.

(2)The main clinical manifestations

It is the usual symptoms of fever, heaviness sensation, dizziness, headache, dysphoria, thirst, stuffiness in the chest, hyperhidrosis, lacking in strength, sickness, bellyache and diarrhea. The tongue body is red and the pulse is full-and-large or rolling-and-rapid.
2. Diagnostic criteria of modern medicine

The low-grade heatstroke:

It is the usual symptoms such as: red face, hot skin, hyperhidrosis, rapid pulse, sickness, vomit and headache. The patients attacked by the heatstroke can't continue the work and the temperature rises up than 38° C.

(II) the verification method

The instant effect should be observed after taking the capsule of the invention on the dosage of 2 capsules once time. If the symptoms had not disappeared after taking the capsules, the patients should take 2 capsules 6 hours later. The observation should be terminated at the 24th hour. At the usual condition, other medicine is forbidden. If the body temperature is higher than 39° C., the patients should be cooled down on a physical method. If the patients loss the body fluid up than a middle-grade level, they should receive the liquor-support treatment to keep the water electrolyte balance. This invented drug is a kind of emergency medicine, so that the instant effect should be observed carefully until the main symptoms disappear. The total course should be less than 24 hours.

(III)The therapeutic evaluation criteria

The instant effect and the change of the main symptoms in 24 hours are the clinical proof to evaluate the curative effect of the invented medicine.
Clinical recovery: all the symptoms have disappeared.
Remarkably effective: all the symptoms have almost disappeared or the main symptoms have disappeared.
Effective: the symptoms have been improved.
Ineffective: the symptoms have not been improved.

(IV)The results:
1.The comparison on the curative effect

In the total 120 patients who received the treatment, 102 patients got clinical recovery, 16 patients got remarkable effective improvement, 2 patients got effective improvement, and no patients felt ineffective. The total effective ratio of the invented medicine was 100%. (see table 5)

TABLE 5
The comparison on the total curative effect(%)

| | | | | | | total |
|---|---|---|---|---|---|---|
| Group | | Clinical- | Remarkably- | | Ineffec- | effec- |
| ratio | Number | recovery | effective | Effective | tive | tive |
| Treat- | 122 | 102 (85%) | 16 (13.33%) | 2 (1.67%) | 0 | 100% |
| ment | | | | | | |

### EXAMPLE 1

| | | | | | |
|---|---|---|---|---|---|
| Camphor | 70 g | dried ginger | 80 g | rhubarb | 40 g |
| Common fennel | 40 g | Chinese cinnamon | 50 g | | |
| hot pepper | 15 g | oleum eucalypti | 100 ml | | |

The dried ginger, Chinese cinnamon, common fennel, rhubarb, hot pepper are crushed into crude powder; the dimension of the powder is 0.5-1 cm; dried ginger, Chinese cinnamon and common fennel are distilled respectively in the distiller to and collect the naphtha for the further use.

The 3 kind of herbs distilled are mixed with rhubarb and hot pepper uniformly and then the mixture are humidified by the 90% alcohol. The humid herbs are percolated by the 80% alcohol 6 times weight of the herbs. The alcohol after the percolation are collected and mixed. The alcohol is recycled from the mixed liquor until no alcohol smell can be felt. The new liquor is condensed in the low pressure to thick paste on the relative density of 1.20 at 60° C. The thick paste is dehydrated in the vacuum and then the dried medicine is crushed into fine powder better than the level of 100-mesh with the water content less than 5%.

The camphor, oleum eucalypti and naphtha are added into the refined vegetable oil and the mixed oil is heated in the water bath at 70° C. until the camphor dissolved. When the oil solution turns clear, the fine powder made from the thick paste is added into the oil. The mixed oil is agitated adequately to prevent the conglomeration of the powder. The mixed oil is adjusted to the right weight by adding refined vegetable oil. The suspended oil which is made uniformly is milled by the colloid mill for 2 times to get a stabilized suspension. At last this suspension is made into 1000 piece of soft. capsules by the soft capsule machine.

### Example II

| | | | | | |
|---|---|---|---|---|---|
| Camphor | 125 g | dried ginger | 125 g | rhubarb | 100 g |
| Common fennel | 50 g | Chinese cinnamon | 50 g | | |
| hot pepper | 25 g | oleum eucalypti | 62.5 ml | | |

The dried ginger, Chinese cinnamon, common fennel, rhubarb, hot pepper are crushed into crude powder; the dimension of the powder is 0.5-1 cm; dried ginger, Chinese cinnamon and common fennel are distilled respectively in the distiller to and collect the naphtha for the further use.

The 3 kind of herbs distilled are mixed with rhubarb and hot pepper uniformly and then the mixture are humidified by the 70% alcohol. The humid herbs are percolated by the 60% alcohol 10 times weight of the herbs. The alcohol after the percolation are collected and mixed. The alcohol is recycled from the mixed liquor until no alcohol smell can be felt. The new liquor is condensed in the low pressure to thick paste on the relative density of 1.20 at 60° C. The thick paste is dehydrated in the vacuum and then the dried medicine is crushed into fine powder better than the level of 100-mesh with the water content less than 5%.

The camphor, oleum eucalypti and naphtha are added into the refined vegetable oil and the mixed oil is heated in the water bath at 60° C. until the camphor dissolved. When the oil solution turns clear, the fine powder made from the thick paste is added into the oil. The mixed oil is agitated adequately to prevent the conglomeration of the powder. The mixed oil is adjusted to the right weight by adding refined vegetable oil. The suspended oil which is made uniformly is milled by the colloid mill for 2 times to get a stabilized suspension. At last this suspension is made into 2000 piece of soft capsules by the soft capsule machine.

1. A pharmaceutical composition for treating heatstroke, characterized in that, it is made from the following materials:

| | |
|---|---|
| camphor | 50-250 part by weight |
| dried ginger | 50-250 part by weight |
| rhubarb | 30-200 part by weight |
| common fennel | 15-100 part by weight |
| chinese cinnamon | 15-100 part by weight |
| hot pepper | 10-60 part by weight |
| oleum eucalypti | 20-120 volume/weight. |

2. The pharmaceutical composition according to claim 1, it is made from the following materials:

| | |
|---|---|
| Camphor | 70 part by weight |
| Dried ginger | 80 part by weight |
| Rhubarb | 40 part by weight |
| Common fennel | 40 part by weight |
| Chinese cinnamon | 50 part by weight |
| Hot pepper | 15 part by weight |
| oleum eucalypti | 100 volume/weight. |

3. The pharmaceutical composition according to claim 1 made from the original herbs as following:

| | |
|---|---|
| Camphor | 125 part by weight |
| Dried ginger | 125 part by weight |
| Rhubarb | 100 part by weight |
| Common fennel | 50 part by weight |
| Chinese cinnamon | 50 part by weight |
| Hot pepper | 25 part by weight |
| oleum eucalypti | 62.5 volume/weight |

4. A method of preparing a medicine for treating heatstroke, comprising:

| | | | | | |
|---|---|---|---|---|---|
| Camphor | 70 g | dried ginger | 80 g | rhubarb | 40 g |
| Common fennel | 40 g | Chinese cinnamon | 50 g | | |
| hot pepper | 15 g | oleum eucalypti | 100 ml | | |

crushing the dried ginger, Chinese cinnamon, common fennel, rhubarb and hot pepper into crude powder with the dimension for 0.5-1 cm;
distilling respectively dried ginger Chinese cinnamon and common fennel in the distiller and collect the naphtha for the further use;
mixing the 3 kind of herbs distilled with rhubarb and hot pepper uniformly and then humidifying the mixture with the 90% alcohol;
percolating the humid herbs with the 80% alcohol 6 times weight of the herbs;
collecting and mixing the alcohol after the percolation and recycling the alcohol from the mixed liquor until no alcohol smell can be felt;
condensing the new liquor in the low pressure to thick paste on the relative density of 1.20 at 60° C.;
dehydrating the thick paste in the vacuum and then crushing the dried medicine into fine powder better than the level of 100-mesh with the water content less than 5%;
adding the camphor, oleum eucalypti and naphtha into the refined vegetable oil and heating the mixed oil in the water bath at 70° C. until the camphor dissolved;
adding the fine powder made from the thick paste into the oil when the oil solution being clear;
agitating adequately The mixed oil;
adjusting the mixed oil to the right weight by adding refined vegetable oil;
milling the suspended oil with the colloid mill for 2 times to get a stabilized suspension;
making this suspension into 1000 piece of soft capsules by the soft capsule machine.

5. A method of preparing a medicine for treating heatstroke comprising:

| | | | | | |
|---|---|---|---|---|---|
| Camphor | 125 g | dried ginger | 125 g | rhubarb | 100 g |
| Common fennel | 50 g | Chinese cinnamon | 50 g | | |
| hot pepper | 25 g | oleum eucalypti | 62.5 ml | | |

crushing the dried ginger, Chinese cinnamon, common fennel, rhubarb and hot pepper into crude powder with the dimension for 0.5-1 cm;
distilling respectively dried ginger Chinese cinnamon and common fennel in the distiller and collect the naphtha for the further use;
mixing the 3 kind of herbs distilled with rhubarb and hot pepper uniformly and then humidifying the mixture with the 70% alcohol;
percolating the humid herbs with the 60% alcohol 10 times weight of the herbs;
collecting and mixing the alcohol after the percolation and recycling the alcohol from the mixed liquor until no alcohol smell can be felt;
condensing the new liquor in the low pressure to thick paste on the relative density of 1.20 at 60° C.;
dehydrating the thick paste in the vacuum and then crushing the dried medicine into fine powder better than the level of 100-mesh with the water content less than 5%;
adding the camphor, oleum eucalypti and naphtha into the refined vegetable oil and heating the mixed oil in the water bath at 60° C. until the camphor dissolved;
adding the fine powder made from the thick paste into the oil when the oil solution being clear;
agitating adequately The mixed oil;
adjusting the mixed oil to the right weight by adding refined vegetable oil;
milling the suspended oil with the colloid mill for 2 times to get a stabilized suspension;
making this suspension into 2000 piece of soft capsules by the soft capsule machine.

6. The use of the pharmaceutical composition according to claim 1, 2 or 3 in the manufacture of a medicament for treating diarrhea.

7. The use of the pharmaceutical composition according to claim 1, 2 or 3 in the manufacture of a medicament for improving sleep.
